(19) United States
(12) Patent Application Publication
(10) Pub. No.: US 2011/0065804 A1
(43) Pub. Date: Mar. 17, 2011

(54) ELECTRODEPOSITED METALLIC FINISHES INCLUDING ANTIMICROBIAL AGENTS

(75) Inventors: Leonard L. Diaddario, Charlotte, NC (US); Matthew W. Stauffer, Fort Mill, SC (US)

(73) Assignees: PAVCO INC., Charlotte, NC (US); MICROBAN INTERNATIONAL LTD., Huntersville, NC (US)

(21) Appl. No.: 12/934,076

(22) PCT Filed: Mar. 25, 2009
(86) PCT No.: PCT/US09/38256

§ 371 Date: Nov. 23, 2010

Related U.S. Application Data
(60) Provisional application No. 61/039,405, filed on Mar. 25, 2008.

(51) Int. Cl. A01N 35/06; A61K 31/05; A01N 31/08; A01P 1/00; C25D 5/00; C25D 5/10; C25D 5/12; C25D 3/12; C25D 3/10; C25D 3/22; C25D 3/32; C25D 3/38

(52) U.S. Cl. 514/690; 514/731; 514/736; 205/80; 205/170; 205/177; 205/178; 205/181; 205/182; 205/271; 205/290; 205/311; 205/302; 205/296; 205/269

(57) ABSTRACT

Articles having metallic finishes including antimicrobial agents dispersed throughout the finish and methods of electroplating said metallic finishes on a material. The metallic finishes include highly-decorative electroplated finishes for bathroom and kitchen hardware, door hardware, and other highly lustrous products where antimicrobial protection is preferred.

### FIELD

The present invention generally relates to metallic finishes including antimicrobial agents dispersed throughout the finish and methods of electroplating said metallic finishes onto a material. The metallic finishes include highly-decorative electroplated finishes for bathroom and kitchen hardware, door hardware, and other highly lustrous products where antimicrobial protection is preferred.

### BACKGROUND

Antimicrobial chemicals can be found in numerous consumer products. Examples of these products include soaps, dental care products, deodorants, other personal care products, first aid products, kitchenware, computer equipment, clothes, children's toys, paints, caulking, and vacuum cleaners. In these examples, the antimicrobial agents are easily incorporated into the product by blending the agents into the formulation.

Recently there has been a trend to develop metal and polymeric (e.g., plastic) materials possessing an antimicrobial property. It is known that antimicrobial properties may be added to stainless steel sheets by sputtering or incorporating silver in the stainless steel. Furthermore, silver ions have been widely used as a disinfectant in the form of silver nitrate. That said, generally, the incorporation of silver into a metal object or article itself has the disadvantage that the metal's characteristics are altered by the inclusion of the silver as well as the disadvantage of the high cost of the metals themselves.

Thermoset resin compositions including antimicrobial compounds have also been developed as coating materials for various metals such as iron, aluminum, copper, and stainless steel. Disadvantageously, the resin compositions include particulate materials such as zeolites and oxides which may be undesirable materials on the surface of articles, e.g., decorative or functional articles, which have high aesthetic requirements.

Although there is some demand for antimicrobial protection in articles with functional metallic coatings, the largest untapped market for antimicrobial protection is in the highly-decorative electroplated finishes market including, but not limited to, bathroom and kitchen plumbing, door hardware, shopping carts, etc. These articles are generally plated in nickel and/or a zinc-nickel alloy with an overlying thin chromium and/or tin-cobalt alloy layer to seal the pores and to prevent oxidation of the underlying layer. For articles that are electroplated with various decorative metallic finishes, the antimicrobial agent must be made to co-deposit with the electroplated metal.

To the inventor's knowledge, to date, no one has successfully electroplated an article whereby at least one of the plated layers includes at least one organic-based antimicrobial agent dispersed therein. The metallic coating including the antimicrobial agent preferably satisfies the high aesthetic standards required for decorative finishes including, but not limited to, high luster, low corrosion, low tarnish and high hardness. In a preferred embodiment, the antimicrobial agent is an organic compound and is uniformly dispersed throughout the electroplated metallic finish.

### SUMMARY

The present invention generally relates to metallic finishes including antimicrobial agents dispersed throughout the finish and methods of electroplating said metallic finishes onto a material.

In one aspect, an article comprising a substrate and a first metallic layer is disclosed, wherein said first metallic layer comprises at least one antimicrobial agent dispersed throughout said first metallic layer. The article may further comprise at least one additional metallic layer, wherein said additional metallic layer comprises at least one antimicrobial agent dispersed throughout said additional metallic layer. It should be appreciated that the additional metallic layer may be directly on the first metallic layer or alternatively, at least one layer substantially devoid of at least one antimicrobial agent may be deposited between the first metallic layer and the additional metallic layer.

In another aspect, an antimicrobial electroplating bath composition is disclosed, said composition comprising a plating composition specific to a metallic layer to be deposited, at least one antimicrobial agent, optionally at least one surfactant, optionally at least one alkali metal salt, and optionally at least one organic solvent.

In yet another aspect, a kit is disclosed, said kit comprising, in one or more containers, one or more of the following reagents for forming an antimicrobial electroplating bath composition, wherein said antimicrobial electroplating bath composition comprises a plating composition specific to a metallic layer to be deposited, at least one antimicrobial agent, optionally at least one surfactant, optionally at least one alkali metal salt, and optionally at least one organic solvent, and wherein the kit is adapted to form the antimicrobial electroplating bath composition suitable for plating a substrate with the metallic layer.

In still another aspect, a method of making an article comprising a first metallic layer having antimicrobial properties dispersed throughout said first metallic layer is disclosed, said method comprising electroplating a substrate with the first metallic layer using an antimicrobial plating bath formulated for the electrodeposition of said first metallic layer, wherein the antimicrobial plating bath comprises a plating composition specific to the first metallic layer to be deposited, at least one antimicrobial agent, optionally at least one surfactant, optionally at least one alkali metal salt, and optionally at least one organic solvent.

In another aspect, an article is described herein, said article comprising a substrate and a first metallic layer, wherein said first metallic layer comprises at least one antimicrobial agent dispersed throughout said first metallic layer, wherein the first metallic layer comprises a metal selected from the group consisting of chromium, nickel, zinc, tin, cobalt, copper, and alloys and combinations thereof, and wherein the antimicrobial agent comprises 5-chloro-2-(2,4-dichlorophenoxy)phenol.

Another aspect relates to an antimicrobial electroplating bath composition comprising a plating composition specific to a metallic layer to be deposited, at least one organic antimicrobial agent, at least one surfactant, optionally at least one alkali metal salt, and optionally at least one organic solvent.

Other aspects, features and advantages of the invention will be more fully apparent from the ensuing disclosure and appended claims.

### DETAILED DESCRIPTION, AND PREFERRED EMBODIMENTS THEREOF

The present invention relates generally to articles including at least one electroplated metallic finish having at least one antimicrobial agent dispersed throughout said finish and a method of making same. Specifically, a multilayer metal-plated article is disclosed, said article having at least one organic antimicrobial agent dispersed throughout at least one of the metallic layers. The multilayer metal-plated article may be useful for a variety of decorative purposes including, but not limited to, bathroom and kitchen hardware and door hardware.

As defined herein, “antimicrobial” is equivalent to antibacterial, antifungal, antiviral, antiparasitic, microbicidal, and microbistatic. As it is known, most antimicrobial agents control microorganism growth by penetrating the microorganisms thin cellular walls, thereby interrupting the organism metabolic function, and finally killing said organism.

As defined herein, an article having “antimicrobial properties” includes any material that kills or inhibits growth of a microorganism. For example, when subjected to the Kirby-Bauer disc diffusion test, microbes should be killed, or their growth retarded, at least below the disc of the material, and most preferably has a measurable zone of inhibition in the immediate area around the disc of the material. In the alternative to the Kirby-Bauer disc diffusion test, however, other antimicrobial test methods known to those skilled in the art may be employed, including without limitation AATCC Test Method 100, AATCC Test Method 30 Part III, or JIS Z2801:2000 (amended 2007).

The Kirby-Bauer disc diffusion test includes the placement of antimicrobial-containing discs onto agar gel that has been swabbed with a bacterium. The antimicrobial agent diffuses from the disc into the agar and if the bacterium is killed, retarded, or inhibited by the antimicrobial agent, there will be no growth in the immediate area around the disc, which is termed the “zone of inhibition.” Standardized charts are available to compare the zone sizes relative to the bacterium species to determine the susceptibility of the bacterium to the agent.

As defined herein, a “microorganism” corresponds to bacteria, fungi, archea and protists and most typically the microorganism is unicellular. Common microorganisms include, but are not limited to, Haemophilus influenzae, Streptococcus pneumoniae, Staphylococcus aureus, Pseudomonas aeruginosa, members of Enterobacteriaceae, Methicillin Resistant Staphylococcus aureus, Enterobacter, Klebsiella, Escherichia coli, Other less frequent agents are enterococci, streptococci other than S. pneumoniae, Serratia marcescens, Citrobacter freundii, Acinetobacter sp. and Xanthomonas sp., Legionella pneumophila, Mycobacterium tuberculosis, Proteus, Streptococcus mutans, Lactobacillus casei, Candida albicans, Clostridium botulinum, Clostridium tetani, Listeria monocytogenes, Mycobacterium leprae, Neissaria meningitides, Treponema pallidum, Cryptococcus neoformans, and Micrococcus luteus.

As defined herein, an “organic” antimicrobial agent corresponds to a compound including an element selected from the group consisting of carbon, hydrogen, oxygen, nitrogen, sulfur, phosphorus, halogens (e.g., fluoro, chloro, bromo, iodo) and combinations thereof, wherein the organic antimicrobial agent is substantially devoid of inorganic metal elements prior to introduction into a plating bath described herein. As defined herein, “substantially devoid” corresponds to less than about 2 wt. %, more preferably less than 1 wt. %, even more preferably less than 0.1 wt. %, and most preferably 0 wt. % of the composition or bulk compound, based on the total weight of said composition or bulk compound.

As defined herein, “dispersed throughout” corresponds to the dispersal of a species, e.g., an antimicrobial agent, homogeneously or heterogeneously throughout the finish, at the completion of the electroplating process. For example, the antimicrobial agent may be homogeneously dispersed throughout the finish such that the concentration of antimicrobial agent at the surface is substantially the same as the concentration at any other sampling location in the layer. Heterogeneous dispersal corresponds to more antimicrobial agent at one sampling location in the layer relative to some other sampling location in the layer. For example, at the completion of the electroplating process, there may be more antimicrobial agent at the surface relative to other sampling locations or there may be islands of more concentrated antimicrobial agent throughout the layer. For the purposes of this invention, “dispersed throughout” can also correspond to the presence of the antimicrobial agent only in the outer atomic layers of the metal layer.

As defined herein, an “article” corresponds to the material that includes a substrate and has been coated with one or more metallic layers as described herein. For example, an article may include a substrate, a nickel layer, a copper layer and a chromium layer.

A “substrate” is typically metal and includes at least one metal species selected from the group consisting of iron, zinc, aluminum, nickel, brass, bronze, copper, lead, cadmium, tin and alloys thereof, steel, stainless steel, and combinations thereof. Alternatively, the substrate is polymeric, e.g., plastic.

The process of producing highly-decorative electroplated finishes is well known in the art. Typically, a basis metal is bright finished on a substrate, followed by the application of a bright electroplate onto the basis metal. For example, the process of electroplating bright nickel having a mirror-like luster which needs no buffing or coloring before receiving a bright chromium plate is well known in the art and used commercially. The bright chrome finish is expected to remain aesthetically pleasing for the life expectancy of the plated article. Typical basis metals include, but are not limited to, nickel, copper and zinc-nickel alloys. An alternative bright electroplate includes a tin-cobalt alloy.

Electroplating formulations for nickel, zinc-nickel alloy, tin-cobalt alloy, zinc, chromium and copper are well known in the art and commercially available, either as proprietary or non-proprietary formulations. For example, a bright nickel electroplating bath may include Lumina (Pavco™, Charlotte, N.C., USA), a zinc-nickel electroplating bath may include Niclipse (Pavco™), a tin-cobalt electroplating bath may include PavCoTing (Pavco™), a chromium electroplating bath may include Hex-A-Gone (Pavco™), and an acid copper bath may include DeCuRate or Coproplate PC (Pavco™). Preferably, for environmental purposes, the chromium electroplating system is based on the chromium (III) ion rather than the hexavalent chromium system (chromium (VI)), although a chromium (VI) system is contemplated herein. The nickel finish may be a bright nickel or a semi-bright nickel finish. It is to be appreciated by one skilled in the art that the proprietary electroplating bath formulations disclosed herein are not intended to limit the electroplating bath formulations in any way. Put another way, the present invention is suitable for use with any proprietary or non-proprietary nickel, zinc-nickel alloy, tin-cobalt alloy, zinc, chromium, or copper electroplating formulations. Moreover, the methods of using said proprietary or non-proprietary nickel, zinc-nickel alloy, tin-cobalt alloy, zinc, chromium, or acid copper electroplating formulations to electroplate said metal onto a substrate are well known in the art. It should be appreciated by one skilled in the art that the methods of depositing a metallic layer having antimicrobial properties dispersed throughout is not limited to the aesthetic metals but may be used to deposit any metal.

Processes of the invention may be embodied in a wide variety of specific embodiments, as hereinafter more fully described.

In one aspect, a method of making an article comprising at least one metallic layer having antimicrobial properties is disclosed, said method comprising electroplating a substrate with at least one metallic layer wherein the at least one metallic layer includes at least one antimicrobial agent dispersed throughout said layer. Said method includes the use of a formulation of the plating bath specific to the at least one metallic layer to be electrodeposited having antimicrobial agent therein. Accordingly, an antimicrobial plating bath formulated for the electrodeposition of the at least one metallic layer on the substrate is also disclosed, said antimicrobial plating bath including a plating composition specific to the at least one metallic layer to be deposited, at least one antimicrobial agent, optionally at least one surfactant, optionally at least one alkali metal salt, and optionally at least one organic solvent.

Surprisingly, the present inventor discovered that antimicrobial agents can be emulsified in a plating bath and subsequently electrodeposited with various functional or decorative metallic coatings. Depending on the metallic finish to be achieved, e.g., Ni, Cr, Zn—Ni, Zn, Sn—Co or Cu, different surfactant(s), alkali metal salt(s) and/or solvent(s) may be used. In other words, the emulsifying agent/surfactant can be selected as to not affect the aesthetics of the electrodeposited metallic finish.

Useful antimicrobial agents in the present invention are organic, non-toxic, and environmentally safe during their use. The antimicrobial agent should be selected as to not affect the aesthetics of the electrodeposited metallic finish. Antimicrobial agents contemplated for use in the formulations of the present invention include organic compounds, more specifically derivatives of phenol, such as halogenated phenols, bis-phenols, alkyl-substituted phenols, and polyphenols, including, but not limited to, 5-chloro-2-(2,4-dichlorophenoxy)phenol (e.g., triclosan, Microban® Additive B, Microban International Ltd., Huntersville, N.C., USA), 2-phenylphenol sodium salt tetrahydrate, 4-(tert-butyl)-2-(2-hydroxyphenyl)phenol, 4-ethyl-2-(2-hydroxyphenyl)phenol, 4-(4-hydroxybutyl)-2-(2-hydroxyphenyl)phenol, 4-(hydroxymethyl)-2-(2-hydroxyphenyl)anisole, 4-(hydroxymethyl)-2-(2-hydroxyphenyl)phenol, 2-(2-hydroxyphenyl)-4-benzylphenol, 4-ethyl-2-phenylphenol, 2-phenyl-4-propylphenol, 5-Isopropyl-biphenyl-2-ol, 4-[4-(tert-butyl)phenyl]phenol, 4-(1,1-dimethylethyl)-phenylphenol, 3-(4-tert-butylphenyl)phenol, 2-(4-tert-butylphenyl)phenol, (2,4-diphenyl)phenol, (4-tert-butyl, 2-phenyl)phenol, 2-(4-tert-butylphenyl)phenol, 3-phenylphenol, resorcinol, hexylresorcinol, hexachlorophene, parabens, thymol, chlorothymol, parachlorometaxylenol, orthophenylphenol, p-tertiary butylphenol, p-tertiaryamylphenol, o-benzylphenyl-p-chlorophenol, parachlorophenol, camphorated parachlorophenol, tetrabromomethylphenol, 2,6-dimethyl-4-chlorophenol, parachlorometaxylenol, and combinations thereof. Most preferably, the antimicrobial agent comprises 5-chloro-2-(2,4-dichlorophenoxy)phenol.

The surfactant(s) used should not substantially affect the aesthetics of the electrodeposited metallic finish. Surfactants contemplated herein include anionic surfactants, non-ionic surfactants, cationic surfactants and combinations thereof, preferably anionic and/or non-ionic surfactants. Anionic surfactants include fluorosurfactants, sodium alkyl sulfates, ammonium alkyl sulfates, alkyl (C₁₀-C₁₈) carboxylic acid ammonium salts, sodium sulfosuccinates and esters thereof, alkyl (C₁₀-C₁₈) sulfonic acid sodium salts, R₁ benzene sulfonic acids or salts thereof (where the R₁ is a straight-chained or branched C₈-C₁₈ alkyl group), sodium alkyl phosphates, ammonium alkyl phosphates, and combinations thereof, such as, but not limited to, dihexylsulfosuccinate sodium salt, dioctyl sodium sulfosuccinate, sodium xylene sulfonate, dodecylbenzenesulfonic acid, sodium dodecyl sulfate, di-anionic sulfonates, and combinations thereof. Non-ionic surfactants include fluoroalkyl surfactants, ethoxylated fluorosurfactants, polyethylene glycols, polypropylene glycols, polyethylene-polypropylene block co-polymers, polyethylene or polypropylene glycol ethers, dinonylphenyl polyoxyethylene, silicone or modified silicone polymers, acetylenic diols or modified acetylenic diols, alkylphenol ethoxylates, naphthol ethoxylates and derivatives thereof, polyacrylate polymers, and combinations thereof, such as sulfopropylated 2-napthol ethoxylate (Ralufon NAPE 14-90, RASCHIG Corporation, Oak Park, Ill., USA), Berol®226 (Akzo Nobel Surfactants, Chicago, Ill., USA), alpha-(nonylphenyl-omega-hydroxy-poly(oxy-1,2-ethanediyl) (Surfonic® N-95, Huntsmen Petrochemical Corporation, Houston, Tex., USA), and ethoxylated β-naphthol.

Organic co-solvents contemplated for use include, but are not limited to, glycol ethers, alcohols, diols, and combinations thereof, such as methanol, ethanol, isopropanol, butanol, tert-butyl alcohol, ethylene glycol, propylene glycol, diethylene glycol, dipropylene glycol, 1,3-butanediol, diethylene glycol monomethyl ether, triethylene glycol monomethyl ether, diethylene glycol monoethyl ether, triethylene glycol monoethyl ether, ethylene glycol monopropyl ether, ethylene glycol monobutyl ether, diethylene glycol monobutyl ether (i.e., butyl carbitol), triethylene glycol monobutyl ether, ethylene glycol monohexyl ether, diethylene glycol monohexyl ether, ethylene glycol phenyl ether, propylene glycol methyl ether, dipropylene glycol methyl ether, tripropylene glycol methyl ether, dipropylene glycol dimethyl ether, dipropylene glycol ethyl ether, propylene glycol n-propyl ether, dipropylene glycol n-propyl ether (DPGPE), tripropylene glycol n-propyl ether, propylene glycol n-butyl ether, dipropylene glycol n-butyl ether, tripropylene glycol n-butyl ether, propylene glycol phenyl ether, and combinations thereof.

Alkali metal salts contemplated herein include a lithium, sodium, potassium, rubidium or cesium cation, or combinations thereof, with any of the following anions: hydroxide, nitrite, nitrate, sulfite, sulfate, phosphate, phosphite, cyanide, carbonate, bicarbonate, acetate, oxide, sulfide, nitride, phosphide, halide (e.g., fluoride, chloride, bromide, iodide), or combinations thereof. Preferably, the alkali metal salt is completely dissolved in the antimicrobial plating bath described herein.

In practice, the antimicrobial plating bath formulated for the electrodeposition of the at least one metallic layer on the substrate broadly includes a plating composition specific to the at least one metallic layer to be deposited, at least one antimicrobial agent, optionally at least one surfactant, optionally at least one alkali metal salt, and optionally at least one organic solvent. As described hereinabove, the “plating composition specific to the at least one metallic layer to be deposited” include proprietary or non-proprietary nickel, zinc-nickel alloy, tin-cobalt alloy, zinc, chromium, acid copper and other metal electroplating formulations, as readily determined by one skilled in the art. In one embodiment, the antimicrobial plating bath comprises, consists of or consists essentially of the plating composition specific to the at least one metallic layer to be deposited, at least one antimicrobial agent and at least one surfactant. In another embodiment, the antimicrobial plating bath comprises, consists of or consists essentially of the plating composition specific to the at least one metallic layer to be deposited, at least one antimicrobial agent, at least one surfactant, and at least one organic solvent. In yet another embodiment, the antimicrobial plating bath comprises, consists of or consists essentially of the plating composition specific to the at least one metallic layer to be deposited, at least one antimicrobial agent, and at least one organic solvent. Another embodiment relates to an antimicrobial plating bath comprising, consisting of or consisting essentially of the plating composition specific to the at least one metallic layer to be deposited, at least one antimicrobial agent, at least one surfactant, and at least one alkali metal salt. Still another embodiment relates to an antimicrobial plating bath comprising, consisting of or consisting essentially of the plating composition specific to the at least one metallic layer to be deposited, at least one antimicrobial agent, at least one surfactant, at least one alkali metal salt, and at least one organic solvent. When present, the at least one surfactant may comprise a species selected from the group consisting of anionic surfactants, non-ionic surfactants, cationic surfactants, and combinations thereof.

In another embodiment, the antimicrobial plating bath comprises, consists of or consists essentially of about 91 wt % to about 99.99 wt % of the plating composition specific to the at least one metallic layer to be deposited, about 0.001 wt % to about 5 wt % (i.e., about 10 ppm to about 50000 ppm) antimicrobial agent, about 0.001 wt % to about 2 wt % surfactant (when present), about 0.001 wt % to about 2 wt % alkali metal salt (when present), and about 0.001 wt % to about 2 wt % organic solvent (when present). Preferably, the amount of antimicrobial agent is in a range from about 50 ppm to about 5000 ppm, more preferably about 100 ppm to about 2000 ppm and most preferably about 100 ppm to about 1000 ppm.

The components of the antimicrobial plating baths are easily formulated by simple addition of the respective ingredients and mixing to homogeneous condition. Furthermore, the antimicrobial plating baths may be readily formulated as single-package formulations or multi-part formulations that are mixed at the point of use. The individual parts of the multi-part formulation may be mixed at the electroplating apparatus or in a storage tank upstream of the electroplating apparatus. Accordingly, another aspect relates to a kit including, in one or more containers, one or more components adapted to form the plating baths described herein. The kit may include, in one or more containers, a proprietary or non-proprietary plating composition specific to the at least one metallic layer to be deposited, at least one antimicrobial agent, optionally at least one organic solvent, optionally at least one alkali metal salt, and optionally at least one surfactant, for later combination. For example, one container may include the proprietary or non-proprietary plating composition specific to the at least one metallic layer to be deposited, another container may include at least one antimicrobial agent, e.g., as a solid, and another container may include the at least one surfactant and/or at least one organic solvent, whereby the contents of the containers are combined by the user at or before the electroplating apparatus. In another alternative, one container may include the proprietary or non-proprietary plating composition specific to the at least one metallic layer to be deposited and the other container may include at least one antimicrobial agent and at least one surfactant, at least one organic solvent or the combination of surfactant(s)/organic solvent(s), whereby the contents of the containers are combined by the user at or before the electroplating apparatus. In still another alternative, one container may include the proprietary or non-proprietary plating composition specific to the at least one metallic layer to be deposited, at least one surfactant and optionally at least one organic solvent, and the other container may include at least one antimicrobial agent, at least one surfactant, at least one alkali metal salt and optionally at least one organic solvent, whereby the contents of the containers are combined by the user at or before the electroplating apparatus. In yet another alternative, one container may include the proprietary or non-proprietary plating composition specific to the at least one metallic layer to be deposited, at least one surfactant and optionally at least one organic solvent, and the other container may include at least one antimicrobial agent, at least one alkali metal salt and optionally at least one organic solvent, whereby the contents of the containers are combined by the user at or before the electroplating apparatus. For kit containers including at least one antimicrobial agent, at least one surfactant, at least one alkali metal salt and/or at least one organic solvent, it should be appreciated that the concentration of antimicrobial agent(s), surfactant(s), organic solvent(s) and/or alkali salt(s) will be higher so that upon dilution, the preferred concentration of each in the antimicrobial plating bath formulation is achieved.

In practice, the method of making an article comprising at least one metallic layer having antimicrobial properties comprises electroplating a substrate with at least one metallic layer using an antimicrobial plating bath formulated for the electrodeposition of said at least one metallic layer, as described herein, wherein the at least one metallic layer includes at least one antimicrobial agent dispersed throughout said layer.

In one embodiment, a method of making an article comprising a nickel layer having antimicrobial properties is disclosed, said method comprising electroplating a substrate with a nickel layer using an antimicrobial plating bath formulation, wherein the antimicrobial plating bath formulation comprises, consists of or consists essentially of a proprietary or non-proprietary nickel plating bath formulation, at least one antimicrobial agent, optionally at least one surfactant, optionally at least one alkali metal salt, and optionally at least one organic solvent, and wherein the nickel layer includes at least one antimicrobial agent substantially dispersed throughout said layer. Preferably, the antimicrobial plating bath formulation comprises, consists of or consists essentially of a proprietary or non-proprietary nickel plating bath formulation, at least one antimicrobial agent, at least one surfactant, optionally at least one alkali metal salt, and optionally at least one organic solvent, and the nickel layer includes at least one antimicrobial agent substantially dispersed throughout said layer. The nickel layer may be a “bright nickel” or a “semi-bright nickel” layer, as understood by one skilled in the art.

In another embodiment, a method of making an article comprising a zinc-nickel layer having antimicrobial properties is disclosed, said method comprising electroplating a substrate with a zinc-nickel layer using an antimicrobial plating bath formulation, wherein the antimicrobial plating bath formulation comprises, consists of or consists essentially of a proprietary or non-proprietary zinc-nickel plating bath formulation, at least one antimicrobial agent, optionally at least one surfactant, optionally at least one alkali metal salt, and optionally at least one organic solvent, and wherein the zinc-nickel layer includes at least one antimicrobial agent substantially dispersed throughout said layer. Preferably, the antimicrobial plating bath formulation comprises, consists of or consists essentially of a proprietary or non-proprietary zinc-nickel plating bath formulation, at least one antimicrobial agent, at least one surfactant, optionally at least one alkali metal salt, and optionally at least one organic solvent, and the zinc-nickel layer includes at least one antimicrobial agent substantially dispersed throughout said layer.

In yet another embodiment, a method of making an article comprising a tin-cobalt layer having antimicrobial properties is disclosed, said method comprising electroplating a substrate with a tin-cobalt layer using an antimicrobial plating bath formulation, wherein the antimicrobial plating bath formulation comprises, consists of or consists essentially of a proprietary or non-proprietary tin-cobalt plating bath formulation, at least one antimicrobial agent, optionally at least one surfactant, optionally at least one alkali metal salt, and optionally at least one organic solvent, and wherein the tin-cobalt layer includes at least one antimicrobial agent substantially dispersed throughout said layer. Preferably, the antimicrobial plating bath formulation comprises, consists of or consists essentially of a proprietary or non-proprietary tin-cobalt plating bath formulation, at least one antimicrobial agent, at least one surfactant, optionally at least one alkali metal salt, and optionally at least one organic solvent, and the tin-cobalt layer includes at least one antimicrobial agent substantially dispersed throughout said layer.

In another embodiment, a method of making an article comprising a chromium layer having antimicrobial properties is disclosed, said method comprising electroplating a substrate with a chromium layer using an antimicrobial plating bath formulation, wherein the antimicrobial plating bath formulation comprises, consists of or consists essentially of a proprietary or non-proprietary chromium plating bath formulation, at least one antimicrobial agent, optionally at least one surfactant, optionally at least one alkali metal salt, and optionally at least one organic solvent, and wherein the chromium layer includes at least one antimicrobial agent dispersed throughout said layer. Preferably, the antimicrobial plating bath formulation comprises, consists of or consists essentially of a proprietary or non-proprietary chromium plating bath formulation, at least one antimicrobial agent, at least one surfactant, optionally at least one alkali metal salt, and optionally at least one organic solvent, and the chromium layer includes at least one antimicrobial agent dispersed throughout said layer.

In still another embodiment, a method of making an article comprising a copper layer having antimicrobial properties is disclosed, said method comprising electroplating a substrate with a copper layer using an antimicrobial plating bath formulation, wherein the antimicrobial plating bath formulation comprises, consists of or consists essentially of a proprietary or non-proprietary acid copper plating bath formulation, at least one antimicrobial agent, optionally at least one surfactant, optionally at least one alkali metal salt, and optionally at least one organic solvent, and wherein the copper layer includes at least one antimicrobial agent dispersed throughout said layer. Preferably, the antimicrobial plating bath formulation comprises, consists of or consists essentially of a proprietary or non-proprietary acid copper plating bath formulation, at least one antimicrobial agent, at least one surfactant, optionally at least one alkali metal salt, and optionally at least one organic solvent, and the copper layer includes at least one antimicrobial agent dispersed throughout said layer.

In still another embodiment, a method of making an article comprising at least two metallic layers having antimicrobial properties is disclosed, said method comprising:
electroplating a substrate with a first metallic layer using a first antimicrobial plating bath formulation, wherein the first antimicrobial plating bath formulation comprises, consists of or consists essentially of a plating composition specific to the first metallic layer to be deposited, at least one antimicrobial agent, optionally at least one surfactant, optionally at least one alkali metal salt, and optionally at least one organic solvent; and
electroplating the substrate having the first metallic layer thereon with at least one additional metallic layer using a second antimicrobial plating bath formulation, wherein the second antimicrobial plating bath formulation comprises, consists of or consists essentially of a plating composition specific to the additional metallic layer to be deposited, at least one antimicrobial agent, optionally at least one surfactant, optionally at least one alkali metal salt, and optionally at least one organic solvent;
   wherein the first and the at least one additional metallic layers may be the same as or different from one another and can include antimicrobial agent(s) dispersed throughout said layers. For example, the first metallic layer may comprise nickel and anti-bacterial agent(s) and a second metallic layer may comprise chromium and anti-bacterial agent(s). Alternatively, the first metallic layer may comprise nickel and anti-bacterial agent(s) and a second metallic layer may comprise a tin-cobalt alloy and anti-bacterial agent(s). It should be appreciated by one skilled in the art that the method may include electroplating the substrate having a second metallic layer thereon with a third metallic layer, etc., wherein the third metallic layer can include a metal that is the same as or different from the metal of the first layer and/or the second layer. For example, the first metallic layer may comprise a zinc-nickel alloy and anti-bacterial agent(s), a second metallic layer may comprise nickel and anti-bacterial agent(s), and a third metallic layer may comprise chromium and anti-bacterial agent(s). It should also be appreciated by one skilled in the art that the plating composition, the at least one antimicrobial agent, the at least one surfactant (when present), the at least one alkali metal salt (when present), and at least one organic solvent (when present) in the first antimicrobial plating bath formulation may be the same as or different from the plating composition, the at least one antimicrobial agent, at least one surfactant (when present), the at least one alkali metal salt (when present) and at least one organic solvent (when present) in the second (third, etc.) antimicrobial plating bath formulation. It should be appreciated that the additional metallic layer may be deposited directly on the first metallic layer or alternatively, at least one layer substantially devoid of antimicrobial agent(s) may be deposited between the first metallic layer and the additional metallic layer.

In yet another embodiment, a method of making an article comprising at least two metallic layers where only one layer has antimicrobial properties at the completion of the electroplating process is disclosed, said method comprising:
electroplating a substrate with a first metallic layer using an antimicrobial plating bath formulation, wherein the antimicrobial plating bath formulation comprises, consists of or consists essentially of a plating composition specific to the first metallic layer to be deposited, at least one antimicrobial agent, optionally at least one surfactant, optionally at least one alkali metal salt, and optionally at least one organic solvent; and
electroplating the substrate having the first metallic layer thereon with at least one additional metallic layer using a second plating bath formulation, wherein the second plating bath formulation comprises a plating composition specific to the second metallic layer to be deposited;
   wherein the first metallic layer includes at least one antimicrobial agent dispersed throughout said layer. Preferably, the second plating bath formulation is substantially devoid of antimicrobial agent. Although not wishing to be bound by theory, it should be appreciated that the at least one antimicrobial agent of the first metallic layer may migrate into the second metallic layer over time.

In another embodiment, a method of making an article comprising at least two metallic layers where only one layer has antimicrobial properties at the completion of the electroplating process is disclosed, said method comprising:
electroplating a substrate with a first metallic layer using a first plating bath formulation, wherein the first plating bath formulation comprises a plating composition specific to the first metallic layer to be deposited; and
electroplating the substrate having the first metallic layer thereon with a second metallic layer using an antimicrobial plating bath formulation, wherein the antimicrobial plating bath formulation comprises, consists of or consists essentially of a plating composition specific to the second metallic layer to be deposited, at least one antimicrobial agent, optionally at least one surfactant, optionally at least one alkali metal salt, and optionally at least one organic solvent; and
   wherein the second metallic layer includes at least one antimicrobial agent dispersed throughout said layer. Preferably, the first plating bath formulation is substantially devoid of antimicrobial agent. Although not wishing to be bound by theory, it should be appreciated that the at least one antimicrobial agent of the second metallic layer may migrate into the first metallic layer over time.

Electroplating processes are well known in the art. For example, proprietary and non-proprietary plating compositions include directions on the favored process of deposition. It is not necessary to alter the process to deposit the antimicrobial plating baths described herein. Between plating steps, the article may be rinsed with water. Subsequent to the deposition of the final metallic layer, the article may be rinsed with water and dried with hot air.

At the completion of any of the method embodiments described herein, an article having at least one metallic layer including at least one antimicrobial agent dispersed throughout the layer will be obtained. Accordingly, another aspect relates to an article comprising at least one metallic layer having at least one antimicrobial agent dispersed throughout said layer. In one embodiment, the article comprises at least two metallic layers, wherein both metallic layers have at least one antimicrobial agent dispersed throughout. In another embodiment, the article comprises at least two metallic layers, wherein only one metallic layer has antimicrobial agent dispersed throughout. Although not wishing to be bound by theory, it should be appreciated that the at least one antimicrobial agent of one metallic layer may migrate into a second metallic layer over time. In a preferred embodiment, the surface of the article is substantially devoid of thermoset resin compositions.

Although not wishing to by theory, it is thought that the antimicrobial agent in the metallic layer migrates to the exposed surfaces of the metallic layer when the agent at the metallic layer surface has been depleted. Furthermore, it is assumed that the antimicrobial agent from the underlying metallic layers (when present) will migrate to the outermost layer when the agent at the outermost metallic layer surface has been depleted.

It should be appreciated that anti-microbial agents may be incorporated into other metallic layers, such as chromium based conversion coatings, according to the methods described herein.

The features and advantages of the invention are more fully shown by the illustrative examples discussed below.

### Example 1

Various antimicrobial plating bath formulations were tested, first for aesthetics of the functional or decorative metallic finish, and if aesthetically pleasing, antimicrobial testing was performed using the Kirby-Bauer test for antibiotic susceptibility.

With regards to the Kirby-Bauer test, Klebsiella pneumoniae or Staphylococcus aurreus were swabbed onto the agar gel and pre-cut discs of the articles prepared herein according to the methods described herein were placed on top of the gel. If the disc has (diffusible) antimicrobial agent therein, the antimicrobial agent will diffuse from the disc into the agar gel. If the bacterial organisms are killed, retarded or inhibited by the antimicrobial agent, there will be no growth in the immediate area below and most preferably around the disc. For the purposes of this invention, no growth under the disc is considered a positive result, although a measurable zone of inhibition (ZOI) in the immediate area around the disc is preferred.

Proprietary metal plating baths were used including, Hex-A-Gone (Pavco™) for chromium, Lumina (Pavco™) for nickel, Niclipse (Pavco™) for zinc-nickel alloy and PavCoTing (Pavco™) for tin-cobalt alloy. The antimicrobial agent used was Microban® Additive B and the surfactant and/or organic solvent used are indicated in Tables 1 and 3. Referring to Tables 1 and 2, tests A-U include the deposition of a first layer including Ni and the deposition of a second layer including Cr, and tests V-Y include the deposition of a first layer including ZnNi, the deposition of a second layer including Ni, and the deposition of a third layer including Cr. Referring to Tables 3 and 4, tests AA-AC include the deposition of a first layer including Ni and the deposition of a second layer including SnCo. In each test described herein, the Microban® Additive B was combined with at least one surfactant and/or at least one organic solvent to form the antimicrobial solution, and a portion of the antimicrobial solution was combined with the plating composition specific to the metallic layer to be deposited such that the ppm of Microban® Additive B stated in Tables 1 and 3 is achieved, as readily determinable by one skilled in the art. A summary of the experiments, as well as the Kirby-Bauer tests are provided in Tables 1 and 3 and Tables 2 and 4, respectively.

TABLE 1
Tests for plating with ZnNi, Ni and Cr

| | | MAB | MAB | MAB | |
|---|---|---|---|---|---|
| | | in ZnNi | in Ni | in Cr | |
| Test | Plate | bath/ppm | bath/ppm | bath/ppm | Antimicrobial solution |
| A | Ni/Cr | — | 40 | 40 | 1 g MAB |
| B | Ni/Cr | — | 200 | 200 | 30 g EGBE |
| C | Ni/Cr | — | 400 | 400 | diluted to 100 mL with DIW |
| D | Ni/Cr | — | 40 | 40 | 1 g MAB |
| E | Ni/Cr | — | 200 | 200 | 50 g EGBE |
| F | Ni/Cr | — | 400 | 400 | 10 g PolyPol 3350 |
| | | | | | 25 g SXS |
| | | | | | diluted to 100 mL with DIW |
| G | Ni/Cr | — | 400 | 400 | 1 g MAB |
| | | | | | 10 g Gemtex ® 680 |
| | | | | | diluted to 100 mL with DIW |
| H | Ni/Cr | — | 0 | 1048 | pyrithione |
| I | Ni/Cr | — | 0 | 80 | 1 g MAB |
| J | Ni/Cr | — | 0 | 160 | 5 g Surfonic ® N-95 |
| K | Ni/Cr | — | 0 | 320 | diluted to 100 mL with DIW |
| L | Ni/Cr | — | 0 | 80 | 1 g MAB |
| M | Ni/Cr | — | 0 | 160 | 10 g EBN |
| | | | | | diluted to 100 mL with DIW |
| N | Ni/Cr | — | 0 | 327 | 1 g MAB |
| | | | | | 25 g Ralufon NAPE 14-90 |
| | | | | | diluted to 100 mL with DIW |
| O | Ni/Cr | — | 0 | 324 | 1 g MAB |
| P | Ni/Cr | — | 41 | 324 | 50 g EGBE |
| | | | | | diluted to 100 mL with DIW |
| Q | Ni/Cr | — | 80 | 320 | 1 g MAB |
| | | | | | 25 g Berol ® 226 |
| | | | | | diluted to 100 mL with DIW |
| R | Ni/Cr | — | 0 | 201 | 10 g MAB |
| S | Ni/Cr | — | 0 | 402 | 500 g PG |
| T | Ni/Cr | — | 201 | 402 | 250 g SXS |
| U | Ni/Cr | — | 401 | 402 | diluted to 1000 mL with DIW |
| | | | | | Added Blancol ®-L to Ni to |
| | | | | | solubilize precipitate |
| V | ZnNi/Ni/Cr | 200 | 0 | 0 | 1 g MAB |
| W | ZnNi/Ni/Cr | 400 | 0 | 0 | diluted to 100 mL with |
| X | ZnNi/Ni/Cr | 400 | 0 | 200 | EGBE |
| Y | ZnNi/Ni/Cr | 400 | 0 | 400 | |

MAB = Microban ® Additive B
EGBE = ethylene glycol monobutyl ether
DIW = deionized water
SXS = sodium xylene sulfonate
EBN = ethoxylated β-naphthol 13-mol EO (ethylene oxide)
PG = propylene glycol

TABLE 2
Kirby-Bauer Results for tests A-Y

| | Klebsiella pneumoniae | | Staphylococcus aureus | |
|---|---|---|---|---|
| Test | Result^{†} | ZOI (mm) | Result^{†} | ZOI (mm) |
| A | inhibition | 3 | inhibition | 3 |
| B | inhibition | 5 | inhibition | 5 |
| C | inhibition | 5 | inhibition | 5 |
| D | no inhibition | — | no inhibition | — |
| E | inhibition | 4 | inhibition | 4 |
| F | inhibition | 5 | inhibition | 10 |
| G | no inhibition | — | no inhibition | — |
| H | inhibition | under sample | no inhibition | — |
| I | inhibition | under sample | no inhibition | — |
| J | inhibition | under sample | no inhibition | — |
| K | inhibition | 9 | inhibition | 7 |
| L | no inhibition | — | inhibition | under sample |
| M | no inhibition | — | no inhibition | — |
| N | no inhibition | — | no inhibition | — |
| O | inhibition | under sample | no inhibition | — |
| P | no inhibition | — | no inhibition | — |
| Q | no inhibition | — | no inhibition | — |
| R | no inhibition | — | no inhibition | — |
| S | inhibition | 5 | no inhibition | — |
| T | inhibition | 10 | inhibition | 4 |
| U | inhibition | 4 | inhibition | 2 |
| V | no inhibition | — | inhibition | 5 |
| W | inhibition | 10 | inhibition | 5 |
| X | inhibition | 8 | inhibition | 4 |
| Y | inhibition | 7 | inhibition | 4 |

^{†}In each case, the control, which corresponds to a plating composition specific to the metallic layer to be deposited without the antimicrobial solution was tested as well, none of which resulted in any inhibition of the growth of microorganisms.

TABLE 3
Tests for plating with Ni and SnCo

| | | MAB | MAB | |
|---|---|---|---|---|
| | | in Ni | in SnCo | |
| Test | Plate | bath/ppm | bath/ppm | Antimicrobial solution |
| AA | Ni/SnCo | 0 | 160 | 1 g MAB |
| AB | Ni/SnCo | 0 | 400 | 10 g EBN |
| AC | Ni/SnCo | 80 | 400 | diluted to 100 mL with DIW |

MAB = Microban ® Additive B
DIW = deionized water
EBN = ethoxylated β-naphthol 13-mol EO

TABLE 4
Kirby-Bauer Results for tests AA-AC

| | Klebsiella pneumoniae | | Staphylococcus aureus | |
|---|---|---|---|---|
| Test | Result^{†} | ZOI (mm) | Result^{†} | ZOI (mm) |
| A | inhibition | under sample | no inhibition | — |
| B | inhibition | under sample | inhibition | under sample |
| C | inhibition | under sample | inhibition | under sample |

^{†}In each case, the control, which corresponds to a plating composition specific to the metallic layer to be deposited without the antimicrobial solution was tested as well, none of which resulted in any inhibition of the growth of microorganisms.

A review of the results can be summarized as follows: the at least one antimicrobial agent is electroplated on the sample when specific classes of surfactant and/or solvent are used and there may be a maximum useful concentration of antimicrobial agent whereby the addition of more antimicrobial agent to the electroplating bath will not result in additional antimicrobial protection. In addition, the antimicrobial agent may have the ability to migrate from an underlying layer to the surface of the outermost layer (see, e.g., tests V and W). As such, it can be assumed that the antimicrobial agent in the underlying metallic layer(s) migrates to the exposed surfaces of the outermost metallic layer when the agent at the outermost metallic layer surface has been depleted or was never initially present at all.

Although various illustrative embodiments and features have been disclosed herein, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

1. An antimicrobial electroplating bath composition comprising a plating composition specific to a metallic layer to be deposited, at least one organic antimicrobial agent, optionally at least one surfactant, optionally at least one alkali metal salt, and optionally at least one organic solvent.

2. (canceled)

3. The antimicrobial electroplating bath composition of claim 1, wherein the metallic layer comprises a metal selected from the group consisting of chromium, nickel, zinc, tin, cobalt, copper, and alloys and combinations thereof.

4. The antimicrobial electroplating bath composition of claim 1, wherein the at least one antimicrobial agent comprises an organic compound selected from the group consisting of halogenated phenols, bis-phenols, alkyl-substituted phenols, polyphenols, and combinations thereof.

5. The antimicrobial electroplating bath composition of claim 1, wherein the at least one antimicrobial agent comprises an organic compound selected from the group consisting of 5-chloro-2-(2,4-dichlorophenoxy)phenol, 2-phenylphenol sodium salt tetrahydrate, 4-(tert-butyl)-2-(2-hydroxyphenyl)phenol, 4-ethyl-2-(2-hydroxyphenyl)phenol, 4-(4-hydroxybutyl)-2-(2-hydroxyphenyl)phenol, 4-(hydroxymethyl)-2-(2-hydroxyphenyl)anisole, 4-(hydroxymethyl)-2-(2-hydroxyphenyl)phenol, 2-(2-hydroxyphenyl)-4-benzylphenol, 4-ethyl-2-phenylphenol, 2-phenyl-4-propylphenol, 5-Isopropyl-biphenyl-2-ol, 4-[4-(tert-butyl)phenyl]phenol, 4-(1,1-dimethylethyl)-phenylphenol, 3-(4-tert-butylphenyl)phenol, 2 (4-tert-butylphenyl)phenol, (2,4-diphenyl)phenol, (4-tert-butyl, 2-phenyl)phenol, 2-(4-tert-butylphenyl)phenol, 3-phenylphenol, resorcinol, hexylresorcinol, hexachlorophene, parabens, thymol, chlorothymol, parachlorometaxylenol, orthophenylphenol, p-tertiary butylphenol, p-tertiaryamylphenol, o-benzylphenyl-p-chlorophenol, parachlorophenol, camphorated parachlorophenol, tetrabromomethylphenol, 2,6-dimethyl-4-chlorophenol, parachlorometaxylenol, and combinations thereof.

6. (canceled)

7. The antimicrobial electroplating bath composition of claim 1, comprising at least one surfactant selected from the group consisting of fluorosurfactants, sodium alkyl sulfates, ammonium alkyl sulfates, alkyl (C₁₀-C₁₈) carboxylic acid ammonium salts, sodium sulfosuccinates and esters thereof, alkyl (C₁₀-C₁₈) sulfonic acid sodium salts, R₁ benzene sulfonic acids and salts thereof, sodium alkyl phosphates, ammonium alkyl phosphates, dihexylsulfosuccinate sodium salt, dioctyl sodium sulfosuccinate, sodium xylene sulfonate, dodecylbenzenesulfonic acid, sodium dodecyl sulfate, fluoroalkyl surfactants, ethoxylated fluorosurfactants, polyethylene glycols, polypropylene glycols, polyethylene glycol ethers, polypropylene glycol ethers, dinonylphenyl polyoxyethylene, silicone polymers, modified silicone polymers, acetylenic diols, modified acetylenic diols, polyacrylate polymers, sulfopropylated 2-napthol ethoxylate, alpha-(nonylphenyl-omega-hydroxy-poly(oxy-1,2-ethanediyl), ethoxylated β-naphthol, di-anionic sulfonates, polyethylene-polypropylene block co-polymers, alkylphenol ethoxylates, naphthol ethoxylates and derivatives thereof, and combinations thereof.

8. The antimicrobial electroplating bath composition of claim 1, comprising at least one alkali metal salt comprising at least one cation selected from the group consisting of lithium, sodium, potassium, rubidium, cesium, and combinations thereof, and any anion selected from the group consisting of hydroxide, nitrite, nitrate, sulfite, sulfate, phosphate, phosphite, cyanide, carbonate, bicarbonate, acetate, oxide, sulfide, nitride, phosphide, fluoride, chloride, bromide, iodide, and combinations thereof.

9. The antimicrobial electroplating bath composition of claim 1, comprising at least one organic solvent selected from the group consisting of methanol, ethanol, isopropanol, butanol, tert-butyl alcohol, ethylene glycol, propylene glycol, diethylene glycol, dipropylene glycol, 1,3-butanediol, diethylene glycol monomethyl ether, triethylene glycol monomethyl ether, diethylene glycol monoethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethylene glycol monohexyl ether, ethylene glycol phenyl ether, propylene glycol methyl ether, dipropylene glycol methyl ether, tripropylene glycol methyl ether, dipropylene glycol dimethyl ether, dipropylene glycol ethyl ether, propylene glycol n-propyl ether, dipropylene glycol n-propyl ether (DPGPE), tripropylene glycol n-propyl ether, propylene glycol n-butyl ether, dipropylene glycol n-butyl ether, tripropylene glycol n-butyl ether, propylene glycol phenyl ether, and combinations thereof.

10. An article comprising a substrate and a first metallic layer, wherein said first metallic layer comprises at least one antimicrobial agent dispersed throughout said first metallic layer.

11. The article of claim 10, further comprising a second metallic layer on the first metallic layer.

12. The article of claim 11, wherein said second metallic layer comprises at least one antimicrobial agent dispersed throughout said second metallic layer.

13. The article of claim 10, wherein the first metallic layer comprises a metal selected from the group consisting of chromium, nickel, zinc, tin, cobalt, copper, and alloys and combinations thereof.

14. The article of claim 10, wherein the second metallic layer comprises a metal selected from the group consisting of chromium, nickel, zinc, tin, cobalt, copper, and alloys and combinations thereof.

15. The article of claim 10, wherein the at least one antimicrobial agent comprises an organic compound selected from the group consisting of halogenated phenols, bis-phenols, alkyl-substituted phenols, polyphenols, and combinations thereof.

16.-18. (canceled)

19. The article of claim 11, wherein the first metallic layer and the second metallic layer comprise metals that are the same as or different from one another.

20. The article of claim 10, wherein said first metallic layer comprises at least one antimicrobial agent dispersed throughout said first metallic layer, wherein the first metallic layer comprises a metal selected from the group consisting of chromium, nickel, zinc, tin, cobalt, copper, and alloys and combinations thereof, and wherein the antimicrobial agent comprises 5-chloro-2-(2,4-dichlorophenoxy)phenol.

21.-23. (canceled)

24. A method of making an article comprising a first metallic layer having antimicrobial properties dispersed throughout said first metallic layer, said method comprising electroplating a substrate with the first metallic layer using an antimicrobial plating bath formulated for the electrodeposition of said first metallic layer, wherein the antimicrobial plating bath comprises a plating composition specific to the first metallic layer to be deposited, at least one antimicrobial agent, optionally at least one surfactant, optionally at least one alkali metal salt, and optionally at least one organic solvent.

25. The method of claim 24, further comprising electroplating the first metallic layer with a second metallic layer using an antimicrobial plating bath formulated for the electrodeposition of said second metallic layer, wherein the antimicrobial plating bath comprises a plating composition specific to the second metallic layer to be deposited, at least one antimicrobial agent, optionally at least one surfactant, optionally at least one alkali metal salt, and optionally at least one organic solvent.

26. The method of claim 24, wherein the first metallic layer comprises a metal selected from the group consisting of chromium, nickel, zinc, tin, cobalt, copper, and alloys and combinations thereof.

27. The method of claim 25, wherein the second metallic layer comprises a metal selected from the group consisting of chromium, nickel, zinc, tin, cobalt, copper, and alloys and combinations thereof.

28. The method of claim 24, wherein the at least one antimicrobial agent comprises an organic compound selected from the group consisting of halogenated phenols, bis-phenols, alkyl-substituted phenols, polyphenols, and combinations thereof.

29. The method of claim 25, wherein the second metallic layer has antimicrobial properties dispersed throughout said second metallic layer.
